(19) United States
(12) Patent Application Publication
(10) Pub. No.: US 2008/0255224 A1
(43) Pub. Date: Oct. 16, 2008

(54) PHARMACOLOGICAL TREATMENT OF PSORIASIS

(76) Inventor: Richard S. Blum, East Hills, NY (US)

Correspondence Address: KATTEN MUCHIN ROSENMAN LLP, 575 MADISON AVENUE, NEW YORK, NY, 10022-2585, US

(21) Appl. No.: 11/952,629

(22) Filed: Dec. 7, 2007

Related U.S. Application Data
(60) Provisional application No. 60/923,575, filed on Apr. 16, 2007.

(51) Int. Cl. A61K 31/352; A61P 17/06

(52) U.S. Cl. 514/454

(57) ABSTRACT

The present invention relates generally to the prevention and treatment of psoriasis and psoriatic-related skin conditions via administration of a cannabinoid agonist, especially nabinone, either alone or in combination with other agents that possess anti-psoriatic pharmacological activity.

### RELATED APPLICATIONS

The present application claims priority from U.S. Provisional Application No. 60/923,575, filed Apr. 16, 2007, the disclosure of which is hereby incorporated by reference in its entirety.

### FIELD OF THE INVENTION

The present invention relates to formulations and methods of treating psoriasis and psoriasis-related skin conditions using CB1 and CB2 receptor agonists, pharmaceutically acceptable salts thereof, or derivatives thereof. More specifically, the present invention is directed to a method of treating psoriasis in a patient comprising administering a pharmaceutically effective amount of nabilone in association with a pharmaceutically acceptable carrier and/or excipient that prevents, delays, ameliorates and cures target signs and symptoms of psoriasis in the patient.

### BACKGROUND OF THE INVENTION

Psoriasis is a noncontagious, lifelong skin disease which, according to the National Institutes of Health, afflicts as many as 7.5 million Americans. The most common form, plaque psoriasis, appears as raised, red patches or lesions covered with a silvery white buildup of dead cells, called scale. The normal life cycle of cells is one month before they die and flake off. With psoriasis, the entire life cycle takes only days. As a result, cells build up rapidly, forming thick silvery scales and itchy, dry, red patches that are sometimes painful. The lesions which may be one of five types, plaque, guttate, inverse, pustular or erythrodermic, may occur on any part of the body. (http://www.psoriasis.org/home/-National Psoriasis Foundation).

Present therapy is predominantly aimed at the immune system and the attendant inflammation. Treatment of mild cases involves topical treatments or phototherapy using UVB, PUVA and lasers. Systemic medications and topical calcipitriol, are usually reserved for patients with moderate to severe psoriasis. Several agents, methotrexate and 6-thioguanine, are actually chemotherapeutics used in oncology. These agents are both hepatotoxic and teratogenic. Bone marrow suppression and anemia can occur at any time during treatment. Other systemic agents include retinoids such as isotretinoin (Accutane®, Roche Pharmaceuticals) and acitretin (Soriatane®, Connetics Corporation). Retinoids can cause severe, life-threatening birth defects, psychiatric symptoms, and hepatotoxicity. Additional systemics are glucocorticoids, hydrea, sulfasalazine, cyclosporin and mycophenolate. All the above drugs have numerous drug interactions. Recently, biologics such as infliximab (Remicade®, Centocor), efalizumab (Raptiva®, Genentech), adalimumab (Humira®, Abbott), etanercept (Enbrel®, Amgen) and alefacept (Amevive®, Biogen Idec) have been used to treat psoriasis. Each of these agents requires injections or infusions which must be either given in a physician's office or self-administered by patients themselves. Current antipsoriatic medications are associated with deleterious side effects, morbidity, and rarely, even mortality. Some agents suppress the immune system and long-term effects are not known. Serious infections, increased risk of malignancies, and cardiovascular events are the greatest health concerns with immunosuppressant agents. In some instances, once the drugs are discontinued, the eruption may show a marked exacerbation. None of the therapies are specifically targeted to the itching associated with the disease. No cure exists and for some patients the disease progresses from a nuisance to a disability.

### SUMMARY OF THE INVENTION

The present invention provides a composition and method of treating psoriasis and other psoriasis-related skin disorders comprising administration to a patient in need of such treatment an effective amount of a cannabinoid (CB) agonist.

The invention also provides use of a CB agonist, preferably nabilone, for treatment of psoriasis and other skin disorders at a preferred dosage range of 0.25-3 mg/day, preferably 0.5-1 mg/day.

Nabilone has now been unexpectedly found by the present inventor to act as an antipsoriatic agent in vivo. Accordingly, a first aspect of the invention provides use of nabilone as an anti-psoriatic agent.

According to another aspect, the present invention concerns the use of nabilone for the manufacture of a pharmaceutical composition for the treatment of psoriasis and psoriasis-related skin conditions.

According to still another aspect, the present invention concerns a method of treating psoriasis including administering a pharmaceutical composition comprising an effective amount of a CB agonist, preferably nabilone or pharmaceutically acceptable analogs, derivatives, prodrugs, salts and enantiomers thereof.

According to yet another aspect, the present invention provides a single non-toxic agent for prevention and treatment of psoriasis and psoriasis-related skin disorders, which is a CB agonist which binds to the CB1 receptor to eradicate puritic sensations and stimulates the CB2 receptor, thereby acting as an immunomodulator.

According to yet another aspect, the present invention provides a pharmaceutical composition adapted for the treatment of psoriasis and psoriasis-related skin disorders containing a CB agonist, either alone or in combination with other CB agonists and pharmaceutical agents for the prevention and treatment of psoriasis. The present invention also encompasses the prospect of dosage forms that contain nabilone alone and in other instances nabilone combined with other antipsoriatic agents and dosage regimens.

According to one aspect, the present invention concerns the use of CB1 agonists or a pharmaceutically acceptable analogs, derivatives, prodrugs, salts and enantiomers thereof for the manufacture of a pharmaceutical composition for the treatment of psoriasis and psoriasis-related diseases.

According to another aspect, the present invention concerns the use of nabilone or a pharmaceutically acceptable derivative analogs, derivatives, prodrugs, salts and enantiomers thereof for the manufacture of a pharmaceutical composition for the treatment of psoriasis and psoriasis-related diseases.

The invention is also directed at kits containing a CB agonist for treating psoriasis and psoriatic-related skin conditions.

The present invention satisfies a long-felt need by providing a particularly effective treatment of psoriasis and psoriasis-related skin conditions by the use of nabilone or a pharmaceutically acceptable analogs, derivatives, prodrugs, salts and enantiomers thereof.

An advantage of the use of nabilone is that, as nabilone has already been FDA-approved and used extensively for nausea and vomiting of chemotherapy, and has been subject of extensive toxicology and safety studies, it is known to be well tolerated and does not show immunosuppressive potential.

Another advantage of the use of nabilone is that there is no need for parenteral administration. Yet another advantage is that at the dosages used for prevention and treatment of psoriasis, the side effect profile is very favorable.

Other advantages of the present invention will appear in the following detailed description

Nabilone may be synthesized as in Archer R A, Blanchard W W B, Day W A et al. Cannabinoids. 3. Synthetic approaches to 9-ketocannabinoids. Total synthesis of nabilone. J Org. Chem. 1977 Jun. 24; 42(13):2277-84. Intermediates of nabilone include, but are not limited to, 1-(tert-Butyldimethylsiloxy)-3-(2,6-di,ethoxyphenyl)-4-isopropenylcyclohexane. Non-limiting examples of nabilone derivatives which can be used in the present invention are the salts of alkali or alkali-earth metals such as sodium salt, potassium salt, magnesium salt, calcium salt, etc., and ester derived from C₁-C₆ alcohols such as ester derived from methanol, ethanol, etc. However, the use of nabilone is preferred.

### DETAILED DESCRIPTION OF THE INVENTION

Recent research with synthetic cannabinoids has shown promise in decreasing the condition of neuropathic pain. CB1 receptors are expressed by central and peripheral neurons and CB2 receptors are expressed mainly by immune cells. The sensation of itch (puritus) is carried over nerve fibers in a similar fashion as pain. The stimulation of the endocannabinoid system via a synthetic cannabinoid exogenously administered, and binding to the CB1 receptor eradicates puritic sensations by acting as a neuromodulator. A secondary benefit of the administration of a synthetic cannabinoid is that of stimulation of the CB2 receptor. The CB2 receptor is intimately involved with the immune system, and acts as an immunomodulator. Thus, administration of a single non-toxic agent targets two aspects of the psoriatic disease process and improves the patient's quality of life. The CB agonists useful for the method of the present invention are characterized in being non-specific. That is, they are agonists for both the CB1 and CB2 receptors.

Nabilone (Cesamet®) (Valeant Pharmaceuticals) is a synthetic cannabinoid which has the ability of binding to the CB1 and CB2 receptors. Nabilone is currently available in 1 mg capsules for the treatment of nausea and vomiting in cancer chemotherapy.

In accordance with the present invention, patients suffering from psoriasis or psoriasis-like skin conditions are administered one or more cannabinoid agonists. It is understood that the invention may be useful in any type of psoriasis including: plaque, guttate, inverse, pustular, and erythrodermic. Additionally, it is understood that the CB agonists of the present invention are useful for other types of psoriasis-related skin disorders including, but not limited to, hyperproliferative skin diseases, seborrheic dermatitis, dermatitis, dandruff, and eczema. These diseases can coexist. In some cases, the disease can begin as eczema and over time turn into psoriasis.

In order to use a compound of the present invention or an analog, derivative, prodrug, salt and enantiomer thereof for the treatment and/or prevention of humans and other mammals it is normally formulated in accordance with standard pharmaceutical practice as a pharmaceutical composition. The present invention provides a composition comprising nabilone with a pharmaceutically acceptable carrier and/or excipient. The carrier(s) and/or excipients must be “acceptable” in the sense of being compatible with the compound of the invention and not deleterious to the recipients thereof. Excipients may include fillers, permeabilizing agents, disintegrants, glidants, lubricants, colorants or coloring agents, pH adjusting agents (e.g. fumaric acid, citric acid), binders (e.g. polyethylene glycols, soluble hydroxyalkylcelluloses, polyvinylpyrrolidone, gelatins, natural gums), surfactants (e.g. sorbitan esters, sodium lauryl sulfate), soluble organic salts (e.g. sodium bicarbonate) and the like. Desirably, the excipients are chemically and physically compatible with the active ingredients.

The specific composition is preferably a tablet or capsule form containing between 0.25-2 mg of nabilone, preferably 0.5-1 mg. The dosage ranges from 0.25-3 mg/day, and preferably 0.5-1 mg/day. However, the dosage depends, of course, on the mode of administration. The physician will readily be able to determine doses for patients depending on age, weight, health state and sex of the patient as well as the severity of the disease.

The composition may administered in a single or divided daily dose, preferably one to two doses. The composition can be taken orally for several days, weeks, months or years at any intervals.

The mechanism of nabilone and cannabinoid derivatives in the treatment of psoriasis is not known. However, cannabinoids, compounds acting directly on CB receptors, are currently used for treatment of nausea and vomiting and chronic pain. Cannabinoids are anti-inflammatory and CB receptors are present in human skin. Anadamide, an endogenous CB receptor ligand, inhibits epidermal keratinocyte differentiation. Psoriasis is an inflammatory disease also characterized by epidermal keratinocyte hyperproliferation. Psoriasis is believed to be characterized by a type 1 cytokine pattern; IFN-γ, IL-2, IL-1 and TNF-α are predominantly expressed in this disorder. Cannabinoids have been shown to down regulate the immune system. It has been shown that cannabinoids inhibit lymphocyte proliferation and cytokine production in a range of immune cells, including macrophages/monocytes, lymphocytes. The exposure of macrophages to cannabinoids in vitro or in vivo impairs their functional capabilities. Additionally, cannabinoids decrease TNF-α release, inhibit iNOS transcription, and nitric acid production in macrophages in response to challenge with aerosolized bacterial lipopolysaccharide.

Recently, in vitro keratinocyte proliferation assay tests revealed that CB receptor agonists inhibit keratinocyte proliferation in a concentration-dependent manner. Selective CB2 receptor agonists, JWH015 and BML 190 elicited only partial inhibition, whereas the non-selective CB agonist HU210 produced a concentration-dependent response.

Many compounds, including those identified herein, are CB agonists, and no doubt more will be identified in the future. Other CB agonists or derivatives thereof may also be used in safe and effective amounts. In addition to nabilone, other natural or synthetic cannabinoids, active at the CB1 and/or CB2 receptors may be used in the present invention. Exemplary CB agonists include, but are not limited to, delta 9-tetrahydrocannabinol (delta 9-THC), dronabinol (Marinol®, Solvay Pharmaceuticals), delta 8-THC, Cannabis Sativa, cannabinol, cannabidiol, cannabicyclol, cannabichromene, cannabigerol, dexanabinol, and combinations thereof. Endocannabinoids which are active at CB1 and CB2 receptors, such as 2-arachidonoylglycerol (2 AG) and arachidonoylethanolamide (AEA) may also be used in the present invention. The invention also includes analogs, derivatives, prodrugs, salts, and enantiomers thereof.

As used herein, the term “safe and effective amount” refers to a sufficient amount of a compound, composition or other material to induce prevention, improvement, treatment and amelioration of psoriasis or psoriatic-related skin disorders but low enough to avoid undue side effects (e.g., disorientation), within the scope of sound judgment of the skilled person. The safe and effective amount of the compound, composition or other material may vary with the particular keratinous material being treated, the age and physical condition of the patient being treated, the severity of the skin condition, the duration of treatment, the nature of concurrent therapy, the specific compound, composition, or other material employed, and the factors within the knowledge and expertise of the skilled person. Pruritis may be measured by means known to those skilled in the art. Examples include a visual analogue scale consisting of a 10 cm line ranging from “no itching” (0) to “very itchy” (10).

As used herein, the word “treat”, “treating”, or “treatment” refers to using the compositions of the present invention either prophylactically to prevent outbreaks of psoriasis symptoms, or therapeutically to retard or ameliorate an existing condition characterized by psoriasis or psoriasis-related skin disorders. Also, as used herein, the word “patient” refers to a warm-blooded animal, including a human. Conditions similar to psoriasis also occur in various domestic animals (e.g. mange). The current invention is felt to encompass all similarly involved species.

The invention also relates to a method for therapeutic treatment of psoriasis and psoriatic-related skin conditions. As used herein, the term “psoriasis-related skin disorders” refers to those conditions characterized by epidermal keratinocyte hyperproliferative proliferation; i.e. hyperproliferative skin diseases of whatever type and other conditions including, but not limited to, seborrheic dermatitis, dermatitis, dandruff, and eczema.

The method of treatment of a patient suffering from psoriasis or psoriasis-related skin conditions involves administering to a patient a pharmaceutically acceptable amount of a CB agonist. The preferred CB agonist is nabilone. The patient is preferably a mammal such as a human.

By “pharmaceutically acceptable” such as in the recitation of a “pharmaceutically acceptable salt”, is meant a material that is not biologically or otherwise undesirable, i.e., the material can be incorporated into a pharmaceutical composition administered to a patient without causing any undesirable biological effects or interacting in a deleterious manner with any of the other components of the composition in which it is contained.

According to another embodiment of the present invention, the method of the present invention may involve use of CB agonists as a combination with one or more additional therapeutic agents to be co-administered to a patient to obtain some particularly desired therapeutic end result. It is further understood that the CB agonists of the present invention are useful alone or in combination with a second or more therapeutic agents, that is together with or adjunctive to pharmaceutical compositions including, but not limited to, other antipsoriatic agents, anti-inflammatory agents, anti-bacterials, anti-fungals, antidandruff and antiseborrhetic agents, hyperkeratolytics, agents for lupus, multiform erythema, photo allergic and photo toxic reaction and atopic dermatitis. It is preferred that if a second or more therapeutic agent is used that they both be administered to the patient in synergistic effective amounts. Additionally, the second or more additional therapeutic agent may also be a CB agonist or its pharmaceutically acceptable analogs, derivatives, prodrugs, salts, and enantiomers thereof or one or more anti-psoriatic agent known in the art. More typically, the second and more therapeutic agent will be selected from a different class of therapeutic agents.

According to the method of the present invention, the dosage is such that the undesirable psychotropic effects of CB agonists are minimized or eliminated.

These active agents may be used in amounts usual for a person skilled in the art. Each composition may further comprise, depending on the intended type of application, the constituents conventionally used in the fields under construction which are present in an amount that is suitable for the desired presentation form. It is understood that this invention is not limited to carriers, formulation types, treatment regimens, and so forth, as such may vary. It is also to be understood that the terminology used herein is for the purpose of describing particular embodiments only, and is not intended to be limiting. It must be noted that, as used in this specification and the appended claims, the singular forms “a”, “an” and “the” include plural referents unless the content clearly dictates otherwise.

Since the present invention has an aspect that relates to the treatment of psoriasis and psoriasis-like skin conditions described herein with a combination of ingredients which can be administered separately, the invention also relates to combining separate pharmaceutical compositions in kit form. Combinations of a CB agonist and a second pharmacologic agent for treating psoriasis and psoriatic-related skin conditions may be provided in a kit. The kit would contain, for example, a therapeutically effective amount of nabilone and a pharmaceutically acceptable carrier in a first unit dosage form and a therapeutically effective amount of a second antipsoriatic agent and a pharmaceutically acceptable carrier in a second unit dosage form together with packaging material, where the packaging material comprises a package insert or a label which provides directions for practicing the method. Typically, the kit includes a container containing the separate compositions such as a divided bottle or a divided foil packet. The kit form is particularly advantageous when the separate components are preferably administered in different dosage forms (e.g. tablet and cream), are administered at different dosage intervals, or when titration of the individual components of the combination is desired by the prescribing physician.

Although the invention has been described as orally administered, the invention is not so limited and may be administered topically or by any other suitable means of administration. While oral administration is preferred, transdermal or topical administration may be desirable for patients who are forgetful or petulant about taking oral medicine. The CB agonists of the present invention may also be administered by the sublingual, buccal, percutenaous, intravenous, intramuscular, intranasal or intrarectal route, in particular circumstances. However, the method and route of administration may be any method known in the art, limited by the physical properties of the drugs and the convenience of the patient and the caregiver and is thus not limited to the aforementioned.

A person skilled in the art can select the appropriate presentation form, and also the method of preparing it, on the basis of general knowledge, taking into account the nature of the constituents used and the intended use of the composition. The pharmaceutical preparations of the present invention are manufactured in a manner which is itself well known in the art.

In the present invention, sustained or delayed release technology may be used. For example, when a core comprising the drug is formulated for immediate release, the core can be prepared by any suitable tableting technique known to those skilled in the art. For example, the drug may be admixed with excipient(s) and formed into a tablet core using a conventional tableting press or using conventional wet granulation techniques. However, it is understood that the pharmaceutical preparations may be made by means of conventional mixing, granulating, dragee-making, dissolving, lyophilizing processes. The processes to be used will depend ultimately on the physical properties of the active ingredient used.

In preparing compositions containing cannabinoid agonists, inert pharmaceutically acceptable carriers are used which may either be solid or liquid. Pharmaceutical compositions containing solid form preparations include, but are not limited to, powders, tablets, dispersible granules, capsules, caplets, gums, lozenges, wafers, chewable tablet, films (including muco-adhesive), ovules and liquids such as suspensions, solutions, syrups, and elixers. Liquid formulations may also be prepared by reconstitution of a solid, for example, from a sachet.

A solid carrier can be one or more substances which may also act as diluents, flavoring agents, solubilizers, lubricants, suspending agents, encapsulating material, binders or tablet disintegrating agents. However, any pharmaceutically acceptable carrier may be generally used for this purpose, provided that the carrier does not significantly interfere with the stability or bioavailability of the compounds of this invention.

Liquid form preparations include solutions, suspensions and emulsions. Liquid preparations may be prepared by adding the active component in water with viscous material, i.e. natural or synthetic gums, resins, methylcellulose, sodium carboxymethylcellulose and other well known suspending agents.

The CB agonists of the invention may also be administered topically to the skin or mucosa, either dermally or transdermally. Typical formulations include, but are not limited to, creams, ointments, lotions, gels, hydrogels, pastes, dusting powders, sponges, implants, foams, emulsions, sprays, viscous liquids, shampoos, semisolids, patches, occlusive dressings such as a medicated band-aid or gauze, films, transdermal therapeutic systems or discs which releases the active ingredient at a predetermined rate over a defined period of time to a defined site of application. Liposomes may also be used. Other means of topical administration include delivery by iontophoresis, electroporation, phonophoresis, sonophoresis and needle-free or microneedle injection.

In certain embodiments, in addition to the CB agonist, the topical formulation may further comprise another active ingredient in combination with the CB agonist, e.g. a corticosteroid anti-inflammatory agent.

The CB agonists of the invention may also be administered intranasally or by inhalation, typically in the form of a dry powder inhaler or aerosol or via smoking. Formulations for inhaled/intranasal administration may be formulated to be immediate and/or modified release.

The following examples describe specific aspects of the invention to illustrate the invention and provide a description of the present methods for those skilled in the art. The Examples should not be construed as limiting the invention as the examples merely provide specific methodology useful in the understanding and practice of the invention and its various aspects. While certain preferred and alternative embodiments of the invention have been set forth for purposes of disclosing the invention, modification to the disclosed embodiments can occur to those who are skilled in the art.

All the literature cited herein, where acceptable by the laws of a country or the policies of a patent office, is incorporated by reference.

A person skilled in the art will take care to select the optional additives and/or the amount thereof such that the advantageous properties of the composition according to the invention are not, or are not substantially, adversely affected by the envisaged addition. It is further understood that the other ingredients and adjuvants introduced into the composition must be of a kind and quantity that are not detrimental to the advantageous effect which is sought herein according to the invention.

The following examples describe specific aspects of the invention to illustrate the invention and provide a description of the present methods for those skilled in the art. The Examples should not be construed as limiting the invention as the examples merely provide specific methodology useful in the understanding and practice of the invention and its various aspects.

The following contemplated examples are offered solely for the purpose of illustrating the invention and are not indicated to limit the scope of the invention in any respect.

### EXAMPLE

A patient suffering from psoriasis was treated with oral nabilone on an inpatient, unblinded basis. The patient suffered from psoriasis which had not responded well to prior drug treatments. To assess whether nabilone was beneficial in alleviating symptoms, nabilone at a dose of 1 mg was added to all other anti-inflammatory and anti-psoriatic medications. The results were dramatic. The regimen administered was a 0.5 mg dosage twice daily for four days, and then 1 mg twice daily for five days and eventually 1 mg daily was administered to the patient. Itch which had been intolerable and incessant abolished with the 1^{st} dose. By the 10th day of therapy the skin redness had disappeared.

What is claimed is:

1. A method of treating psoriasis and psoriatic-related skin disorders comprising administering to a patient in need thereof an effective amount of at least one cannabinoid (CB) agonist or its pharmaceutically acceptable analogs, derivatives, prodrugs, salts, and enantiomers thereof.

2. The method of claim 1, wherein the psoriatic-related skin disorders are selected from the group consisting of: hyperproliferative skin disorders, seborrheic dermatitis, dandruff, and eczema.

3. The method of claim 1, wherein the psoriasis is selected from the group consisting of plaque, guttate, inverse, pustular, and erthrodermic.

4. The method of claim 1, wherein the cannabinoid agonist is selected from the group consisting of a CB1 receptor agonist, a CB2 receptor agonist, a mixed CB1/CB2 receptor agonist.

5. The method of claim 4, wherein the CB agonist is a mixed CB1/CB2 receptor agonist.

6. The method of claim 1, wherein the cannabinoid agonist is selected from the group consisting of nabilone, delta 9-tetrahydrocannabinol, dronabinol, delta 8-THC, Cannabis Sativa, cannabinol, cannabidiol, cannabicyclol, cannabichromene, cannabigerol, dexanabinol, arachidonoylethanolamide, 2-arachidonoylglycerol and analogs, derivatives, prodrugs, salts, enantiomers thereof.

7. The method of claim 6, wherein the cannabinoid agonist is nabilone.

8. The method of claim 7, wherein the effective amount of nabilone is between about 0.25 to about 3 mg/day.

9. The method of claim 8, wherein the effective amount of nabilone is between about 0.5 to about 1 mg/day.

10. The method of claim 1, wherein the method of administration is selected from the group consisting of oral, transdermal, topical, sublingual, buccal, percutaneous, intravenous, intramuscular, intranasal, intrarectal.

11. The method of claim 10, wherein the method of administration is oral.

12. The method of claim 11, wherein the method of administration is topically.

13. The method of claim 1, wherein the cannabinoid agonist or its pharmaceutically acceptable analog, derivative, prodrug, salt, and enantiomer is administered with a pharmaceutically-acceptable excipient, diluent or carrier.

14. The method of claim 1, wherein the cannabinoid agonist is administered in combination with another compound or compounds selected from the group consisting of anti-inflammatories, immunosuppressants, and other therapeutic agents for psoriasis and psoriatic-related skin disorders.

15. A pharmaceutical composition for use in treating psoriasis and psoriatic-related skin disorders comprising at least one CB agonist or its pharmaceutically acceptable analogs, derivatives, prodrugs, salts, and enantiomers thereof.

16. The pharmaceutical composition of claim 15, where the cannabinoid agonist is selected from the group consisting of nabilone, delta 9-tetrahydrocannabinol, dronabinol, delta 8-THC, Cannabis Sativa, cannabinol, cannabidiol, cannabicyclol, cannabichromene, cannabigerol, dexanabinol, arachidonoylethanolamide, 2-arachidonoylglycerol and analogs, derivatives, prodrugs, salts, enantiomers thereof.

17. The pharmaceutical composition of claim 16, wherein the cannabinoid agonist is nabilone or analogs, derivatives, prodrugs, salts, and enantiomers thereof.

18. The pharmaceutical composition of claim 14 wherein the at least one cannabinoid agonist is in an amount from about 0.25 to about 2 mg.

19. A kit for treating psoriasis and psoriatic-related skin conditions comprising: a therapeutically effective amount of nabilone; and a pharmaceutically acceptable carrier in a first unit dosage form; and a therapeutically effective amount of a second antipsoriatic agent; and a pharmaceutically acceptable carrier in a second unit dosage form together with packaging material, and wherein said packaging material comprises a package insert or a label which provides directions for practicing the method claimed in claim 13.
